SEQUENCE LISTING (2 sequences)

SEQ ID NO: 1 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 2 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

(19) United States
(12) Patent Application Publication
(10) Pub. No.: US 2011/0008802 A1
(43) Pub. Date: Jan. 13, 2011

(54) TNFalpha GENE EXPRESSION AS A BIOMARKER OF SENSITIVITY TO ANTAGONISTS OF INHIBITOR OF APOPTOSIS PROTEINS

(75) Inventors: Alireza Alavi, Malvern, PA (US); Mark A. McKinlay, Malvern, PA (US); Srinivas K. Chunduru, Malvern, PA (US); John Silke, Victoria (AU); David Vaux, Victoria (AU); James Vince, Victoria (AU)

Correspondence Address: BANNER & WITCOFF, LTD., 1100 13th STREET, N.W., SUITE 1200, WASHINGTON, DC, 20005-4051, US

(73) Assignee: TETRALOGIC PHARMACEUTICALS CORP., Malvern, PA (US)

(21) Appl. No.: 12/598,940

(22) PCT Filed: May 7, 2008
(86) PCT No.: PCT/US2008/062902

§ 371 Date: Apr. 16, 2010

Related U.S. Application Data
(60) Provisional application No. 60/916,426, filed on May 7, 2007.

(51) Int. Cl. G01N 33/53

(52) U.S. Cl. 435/7.21

(57) ABSTRACT

TNFα gene expression can be used as a biomarker of a cell's sensitivity to antagonists of inhibitor of apoptosis proteins (IAPs). Methods of the invention are useful for screening patients to identify those who could benefit from administration of an IAP antagonist to treat various malignant or benign tumors, benign proliferative diseases, or autoimmune diseases.

This application claims the benefit of and incorporates by reference provisional application Ser. No. 60/916,426 filed May 7, 2007.

### FIELD OF THE INVENTION

The invention relates to assaying TNFα gene expression as a biomarker of the sensitivity of an abnormally proliferating cell, including disease-associated cells such as tumor cells and inflammatory cells, to antagonists of inhibitor of apoptosis proteins (IAPs).

### BRIEF DESCRIPTION OF THE FIGURES

FIG. 1 shows a typical CC₅₀ curve for the IAP antagonist compound C in OVCAR-8 cells. “CC₅₀” is the concentration at which 50% of the cells are killed.

FIG. 2 shows a typical CC₅₀ curve for the IAP antagonist compound C in SKOV3 cells.

FIG. 3 is a bar graph showing the average level of TNFα protein secreted into the culture medium over three days by various cell lines, correlated with the cells' response to an IAP antagonist (compound C). The CC₅₀ for each cell line in μM is shown in parentheses on the X-axis.

### SUMMARY OF THE INVENTION

One embodiment of the invention is a method of inducing apoptosis in cells in a cell population. The method comprises assaying a first sample of a cell population in vitro to determine a potential for tumor necrosis factor α (TNFα) gene expression. The cell population is contacted in vitro with an IAP antagonist if TNFα gene expression is detected in the first sample. The cell population can be a cell line.

Another embodiment of the invention is a method of determining sensitivity of cells to an IAP antagonist. The method comprises assaying cells for a potential for TNFα gene expression. The cells are identified as sensitive to an IAP antagonist if TNFα gene expression is detected.

Another embodiment of the invention is a method of predicting sensitivity of abnormally proliferating cells to treatment with an IAP antagonist. The method comprises assaying a sample of abnormally proliferating cells for a potential for TNFα gene expression. The abnormally proliferating cells are identified as sensitive to treatment with an IAP antagonist if the cells express the TNFα gene.

Another embodiment of the invention is a method of inducing apoptosis. The method comprises assaying cells of a cell population to determine a potential for TNFα gene expression. The cell population is contacted with an IAP antagonist if TNFα gene expression is detected.

Another embodiment of the invention is a method of treating a proliferative disorder. The method comprises sampling pathologically proliferating cells obtained from a patient to determine if the cells have a potential for expressing a TNFα gene. An IAP antagonist is administered to the patient if the potential for expressing the TNFα gene is detected. An alternative therapy is administered to the patient if the potential for expressing the TNFα gene is not detected.

Another embodiment of the invention is a method of screening patients for those who could benefit from treatment with an IAP antagonist. The method comprises assaying abnormally proliferating cells obtained from a patient for a potential for TNFα gene expression and determining that the patient would benefit from treatment with an IAP antagonist if expression is detected. In some embodiments the patient is treated with the IAP antagonist.

Another embodiment of the invention is a method of determining sensitivity of cells to an IAP antagonist. The method comprises assaying cells to determine their potential for expressing TNFα gene in response to nuclear factor kappa B (NF-κB). The cells are identified as sensitive to an IAP antagonist if a potential for TNFα gene expression is detected. In some embodiments the potential for expression of the TNFα gene is assayed by determining the presence TNFα mRNA in the cell.

Another embodiment of the invention is a method of determining sensitivity of cells to an IAP antagonist. The method comprises determining if the TNFα gene promoter is methylated. The cells are identified as sensitive to an IAP antagonist if methylation is not detected.

Another embodiment of the invention is a method of determining sensitivity of cells to an IAP antagonist. The method comprises assaying NF-κB response elements within the promoter of the TNFα gene in a sample of cells for mutations. The cells are identified as sensitive to an IAP antagonist if mutations are not detected.

Another embodiment of the invention is the use of an IAP antagonist to treat a proliferative disorder in a patient in whom cells undergoing pathological proliferation are determined to have a potential to express a TNFα gene.

In embodiments described above the cells can be, for example, tumor cells or cells which abnormally proliferate in an autoimmune disorder, including biopsy cells biopsy cells obtained from a patient.

In embodiments described above the cells can be contacted with a cytokine or a growth factor before assaying for TNFα gene expression. Tumor necrosis factor α gene expression can be determined, for example, by detecting TNFα protein. Gene expression can be determined by detecting TNFα mRNA.

In some embodiments described above the IAP antagonist has a binding affinity for at least one of cellular inhibitor of apoptosis protein 1 (cIAP-1) and cellular inhibitor of apoptosis protein 2 (cIAP-2) which is greater than the binding affinity of the IAP antagonist for X-linked inhibitor of apoptosis protein (XIAP). In some embodiments the binding affinity of the IAP antagonist is at least 3-fold greater for cIAP-1 than for XIAP. In other embodiments the binding affinity of the IAP antagonist for cIAP-1 is at least 100 times greater than for XIAP. In still other embodiments the IAP antagonist is a cellular IAP antagonist and an XIAP antagonist.

In some embodiments described above the IAP antagonist has a binding affinity for XIAP that is greater than the binding affinity of the IAP antagonist for at least one of cIAP-1 and cIAP-2.

### DETAILED DESCRIPTION OF THE INVENTION

The invention provides methods of predicting sensitivity of cells to treatment with antagonists of inhibitor of apoptosis proteins (IAP antagonists). A cell is sensitive to an IAP antagonist if it undergoes apoptosis in response to the IAP antagonist. Methods of the invention are useful for predicting which cells are more likely to respond to an IAP antagonist by undergoing apoptosis. The methods can be used either in laboratory or clinical settings.

Methods of the invention are particularly useful for screening patients to identify those who could benefit from administration of an IAP antagonist to treat various benign tumors or malignant tumors (cancer), benign proliferative diseases (e.g., psoriasis, benign prostatic hypertrophy, and restenosis), or autoimmune diseases (e.g., autoimmune proliferative glomerulonephritis, lymphoproliferative autoimmune responses). Cancers which can be treated with IAP antagonists include, but are not limited to, one or more of the following: lung adenocarcinoma, pancreatic-cancer, colon cancer, ovarian cancer, breast cancer, mesothelioma, peripheral neuroma, bladder cancer, glioblastoma, melanoma, adrenocortical carcinoma, AIDS-related lymphoma, anal cancer, bladder cancer, meningioma, glioma, astrocytoma, breast cancer, cervical cancer, chronic myeloproliferative disorders (e.g., chronic lymphocytic leukemia, chronic myelogenous leukemia), colon cancer, endocrine cancers, endometrial cancer, ependymoma, esophageal cancer, Ewing's sarcoma, extracranial germ cell tumors, extragonadal germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, intraocular melanoma, islet cell carcinoma, Kaposi sarcoma, laryngeal cancer, leukemia, acute lymphoblastic leukemia, acute myeloid leukemia, lip cancer, oral cavity cancer, liver cancer, male breast cancer, malignant mesothelioma, medulloblastoma, melanoma, Merkel cell carcinoma, metastatic squamous neck cancer, multiple myeloma and other plasma cell neoplasms, mycosis fungoides and the Sézary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, ovarian epithelial cancer, ovarian germ cell tumors, ovarian low malignant potential tumors, pancreatic cancer, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, skin cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

Some methods of the invention involve assaying cells for TNFα gene expression or for the potential for TNFα gene expression. Cells which express the TNFα gene or which have the potential to express the TNFα gene are sensitive to one or more IAP antagonists. TNFα gene expression can be assayed by any means known in the art. In some embodiments gene expression is assayed by detecting TNFα protein. An amino acid sequence for human TNFα is shown in SEQ ID NO:b 2. TNFα protein (e.g., secreted, contained within a cell, expressed on a cell surface) can be detected, for example, using various immunoassays (ELISA, Western blot, flow cytometry, radioimmunoassays, etc.). In other embodiments gene expression is assayed by detecting TNFα mRNA (e.g., by Northern blot, dot blot, RT-PCR, etc.). A sequence of a human TNFα mRNA is shown in SEQ ID NO:1. A cell which produces any detectable level of TNFα protein or mRNA is a cell which expresses the TNFα gene, although the level of gene expression which can be detected will depend on the assay used.

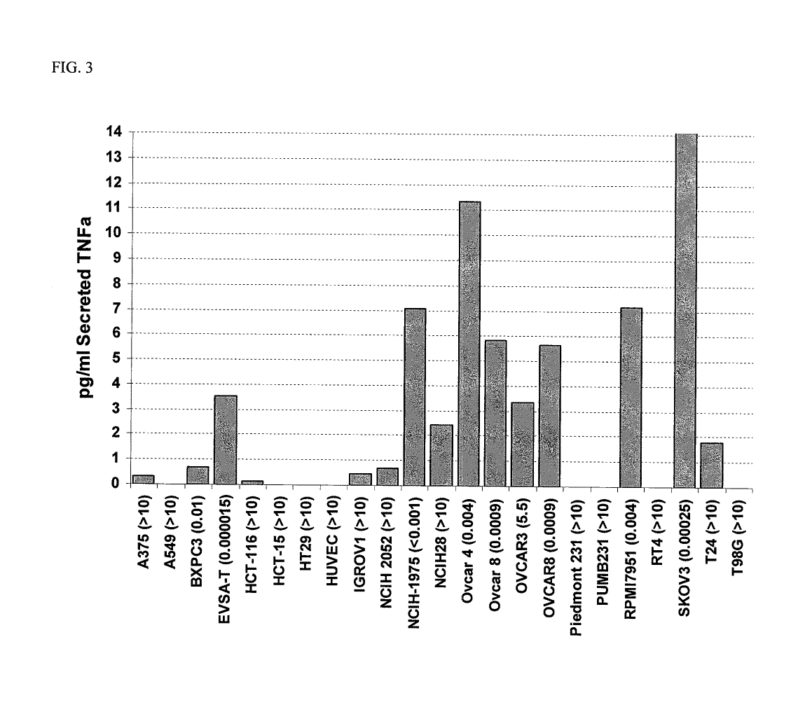

In some embodiments, cells responsive to an IAP antagonist secrete TNFα protein into culture medium at a level higher than about 3 pg/ml. Levels of secreted TNFα in such in vitro assays can range between about 3 pg/ml and about 14 pg/ml (e.g., 3, 4, 5, 6, 7, 8, 9, 10, 11, 12, 13, or 14 pg/ml). Useful time points for collecting and assaying culture medium include 1, 2, and 3 days. See Example 3 and FIG. 3.

Any cell type can be assayed for TNFα gene expression. The cells can be primary cells (e.g., cells of a biopsy obtained from a patient) or cell lines. Of particular interest are cells which proliferate abnormally, including cells which proliferate pathologically and which cause or lead to disease symptoms. Abnormally proliferating cells occur, for example, in cancer, benign proliferative disorders, and autoimmune diseases.

Cells which have the potential to express the TNFα gene in response to NF-κB are sensitive to one or more IAP antagonists. Thus, in some embodiments cells are contacted with a cytokine or growth factor (e.g., interleukin-1, interleukin-6, interferon γ, tumor necrosis factor, or transforming growth factor β) to stimulate the NF-κB pathway before assaying for TNFα gene expression. The ability of a cell to express TNFα in response to a cytokine is predictive of the cell's sensitivity to an IAP antagonist. Potential for expressing the gene can be assayed, for example, by determining the presence of TNFα mRNA in a cell.

Potential for expressing the TNFα gene also can be assessed by determining if the TNFα gene promoter is methylated or by assaying NF-κB response elements within the promoter of the TNFα gene for mutations. The lack of mutations or methylation events within the NF-κB response elements predicts that the cell is likely to undergo apoptosis in response to an IAP antagonist. The presence of methylation or of mutations in the TNFα promoter can indicate that an IAP antagonist will not be effective. Mutations in the TNFα coding sequence can also indicate lack of responsiveness to IAP antagonists.

Some embodiments of the invention include inducing apoptosis of cells, particularly pathologically proliferating cells. The methods can be carried out in vitro or in vivo and can include treatment of a patient with an IAP antagonist. Such treatment can include administration of a single IAP antagonist, administration of a combination of IAP antagonists, or administration of one or more IAP antagonists and one or more chemotherapeutic agents. Administration of multiple agents can be simultaneous or sequential. Useful chemotherapeutic agents include, but are not limited to, alkylating agents (e.g., cyclophosphamide, mechlorethamine, chlorambucil, melphalan), anthracyclines (e.g., daunorubicin, doxorubicin, epirubicin, idarubicin, mitoxantrone, valrubicin), cytoskeletal disruptors (e.g., paclitaxel, docetaxel), epothilones (e.g., epothilone A, epothilone B, epothilone D), inhibitors of topoisomerase II (e.g., etoposide, teniposide, tafluposide), nucleotide analogs precursor analogs (e.g., azacitidine, azathioprine, capecitabine, cytarabine, doxifluridine, fluorouracil, gemcitabine, mercaptopurine, methotrexate, tioguanine), peptide antibiotics (e.g., bleomycin), platinum-based agents (e.g., carboplatin, cisplatin, oxaliplatin), retinoids (e.g., all-trans retinoic acid), and vinca alkaloids and derivatives (e.g., vinblastine, vincristine, vindesine, vinorelbine). In some embodiments, chemotherapeutic agents include fludarabine, doxorubicin, paclitaxel, docetaxel, camptothecin, etoposide, topotecan, irinotecan, cisplatin, carboplatin, oxaliplatin, amsacrine, mitoxantrone, 5-fluoro-uracil, or gemcitabine.

IAP Antagonists

An IAP antagonist for use in the invention is any molecule which binds to and inhibits the activity of one or more IAPs, such as a cellular IAP (cIAP, e.g., cIAP-1 or cIAP-2) or X-linked IAP(XIAP). In some embodiments, an IAP antagonist binds to cIAP-1 and cIAP-2 with greater affinity than it binds to XIAP. In some embodiments, the IAP antagonist binds to cIAP-1 with at least a 3-fold greater affinity than to XIAP, and in others, the IAP antagonist binds to cIAP-1 with at least a 100-fold greater affinity than to XIAP. In still other embodiments, the IAP antagonist is also an XIAP antagonist; some of these antagonists bind to XIAP with greater affinity than to a cIAP. In some embodiments, the IAP antagonist is a mimetic of Smac (second mitochondrial activator of caspases), and in particular embodiments the Smac mimetic is a mimetic or peptidomimetic of the N-terminal 4-amino acids of mature Smac (Ala-Val-Pro-Ile) or, more generally, Ala-Val-Pro-Xaa, wherein Xaa is Phe, Tyr, Ile, or Val, preferably is Phe or Ile.

Many IAP antagonists are known in the art. In certain embodiments, the IAP antagonist is compound A, which has the following structure:

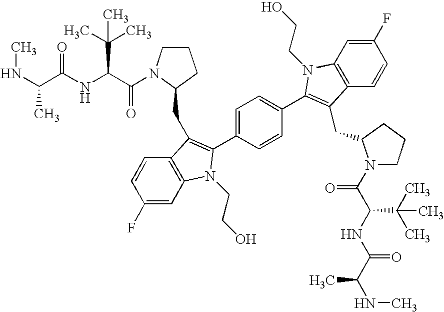

In other embodiments the IAP antagonist is compound C, which has the following structure:

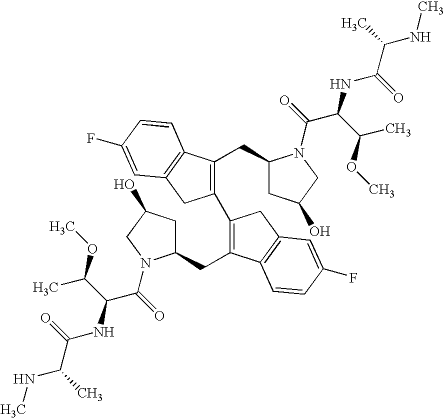

Other examples of IAP antagonists useful in the invention include, but are not limited to, those disclosed in US 2006/0025347; US 2006/0194741; US 2007/0042428; US 2006/0128632; US 2006/0052311; US 2005/0261203; WO 2005/069888; WO 2005/069894; US 2005/0234042; US 2006/0014700; WO 2006/010118; WO 2006/122408; US 2006/0167066; WO 2006/017295; WO 2006/133147; WO 2006/128455; WO 2006/091972; WO 2006/020060; WO 2006/014361; WO 2006/097791; WO 2005/094818; WO 2008/045905; WO 2008/016893; WO 2007/136921; WO 2007/021825; WO 2007/130626; WO 2007/106192; and WO 2007/101347.

In some embodiments pharmaceutical compositions comprising an IAP antagonist are administered to a human or veterinary patient. The pharmaceutical compositions typically comprise a pharmaceutically acceptable carrier or diluent and can be administered in the conventional manner by routes including systemic, topical, or oral routes. For example, administration can be, but is not limited to, parenteral, subcutaneous, intravenous, intramuscular, intraperitoneal, transdermal, oral, buccal, intravaginal, or ocular routes, by inhalation, by depot injections, or by implants. Specific modes of administration will depend on the indication and other factors including the particular compound being administered. The amount of compound to be administered is that amount which is therapeutically effective. The dosage to be administered will depend on the characteristics of the subject being treated, e.g., the particular patient treated, age, weight, health, types of concurrent treatment, if any. Frequency of treatments can be easily determined by one of skill in the art (e.g., by the clinician).

Some embodiments of the invention include a kit for performing the evaluation and analysis of the TNFα promoter or TNFα gene expression. Such kits include Qiagen EPITECT® Bisulfite Conversion Kit followed by TNFα promoter sequencing, antibodies, probes, detectable markers and the like, as well as reagents, gels, apparatuses, analysis tools and so forth necessary to perform the evaluation and analysis of IAP antagonist treatment as described above.

The invention includes methods for marketing IAP antagonists, kits, systems, and methods for using biomarkers useful in determining the likelihood of successful treatment using IAP antagonists. In one embodiment, data regarding the effectiveness of such methods, systems and kits is submitted to a regulatory agency as part of a dossier for seeking approval to conduct human clinical trials with an IAP antagonist, e.g., to establish exclusion or inclusion criteria or to facilitate evaluation of clinical trial data. Such data can be submitted to a regulatory agency to support an application for approval to market a methods, systems, and kits for using biomarkers associated with treatment using IAP antagonists. For example, such data can be submitted as a part of a New Drug Approval Application (NDA) with the United States Food and Drug Administration (FDA).

Various embodiments of the invention include providing information about the responsiveness of cells that are capable of expressing TNFα in response to treatment with an IAP antagonist and disseminating this information to individuals who may be interested in such a pharmaceutical composition comprising an IAP antagonist. Such individuals include those who have a proliferative disorder, medical personnel who treat such disorders, and individuals who dispense or distribute pharmaceuticals.

When approval has been attained for human clinical trials, the previously described information can be included with data supporting the efficacy of pharmaceutical composition on human subjects exhibiting a proliferative disorder, and other data, such as dosage information and cell toxicity data, in a dossier that can be submitted to a regulatory agency for approval to market an IAP antagonist, and pharmaceutical compositions including the IAP antagonist.

Embodiments also include methods for marketing the IAP antagonist or pharmaceutical compositions including the IAP antagonist after approval has been attained. In such methods, information relating to the fact that IAP antagonists are likely to be effective in cells that are capable of expressing TNFα can be disseminated to, for example, physicians, pharmacists, prescribers, insurance providers, distributors, patients, and the like, or combinations of these. In still other embodiments, the information can be disseminated to prospective patients and/or prospective prescribers, and/or prospective distributors.

The information can be disseminated by any method known in the art including, but not limited to, direct-to-consumer advertising, television advertising, radio advertising, newspaper advertising, advertising through printed materials (e.g., pamphlets, leaflets, postcards, letters, and the like), advertising through a web site or on a web site (using for example, a “banner” ad on a web site), billboard advertising, direct mail, e-mail, oral communications, and any combinations thereof.

In other embodiments, the data can be stored in a user accessible database. The data stored in the database can include any data relating to the IAP antagonist or pharmaceutical composition, including, for example, data generated during testing of the methods, systems, and kits for using biomarkers associated with treatment using IAP antagonists, information regarding safety and/or efficacy of the IAP antagonists, pharmaceutical compositions, methods, systems and kits, dosing information, lists of disorders that can be treated using the compound, approval information from one or more regulatory agency, distributor information, prescription information, and combinations thereof.

Various embodiments also include a system for marketing IAP antagonists, pharmaceutical compositions, methods, systems, and kits for using biomarkers associated with treatment using IAP antagonists including a database, such as the database described above, comprising information regarding the methods, systems and kits and data for the efficacy of methods, systems, and kits for using biomarkers associated with treatment using IAP antagonists. In such embodiments, the information held in the database may only be accessible to selected individuals, such as, for example, management personnel, sales personnel, marketing personnel and combinations thereof. The system can also include a subset of the information held in the database that is disseminated to non-selected individuals who can be any person who is not a selected individual, such as, for example, a physician, a pharmacist, a prescriber, an insurance provider, a patient, a distributor and combinations thereof. Dissemination can take place by any dissemination method known in the art as described above.

The subset of data can include any information held in the database and can include information thought to make the methods, systems, and kits marketable, such as, for example, safety and/or efficacy data, lists of disorders that can be treated using the compound, potential side effects of administering the pharmaceutical, list ingredients or active agents in the pharmaceutical composition, approval information from one or more regulatory agency, distributor information, prescription information and combinations thereof. In certain embodiments, the selected individuals can choose and/or approve the information provided in the subset of data.

In each of the embodiments described above, the information provided and/or disseminated and data stored in the database can further include compositions, methods, or protocols for combined therapies that can include another anti-autoimmune or anti-proliferative agent.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### Example 1

### Analysis of IAP antagonist binding to an IAP BIR3 domain

IAPs are characterized by the presence of one or more baculoviral IAP repeats (BIRs).

The BIR domains of XIAP, cIAP-1 and cIAP-2 bind to caspases, the key effector proteases of apoptosis, and XIAP has been shown to be a potent physiological inhibitor of caspase 9 and caspase 3.

However, cIAP-1 and cIAP-2 were originally identified by their association with TNF-R2 via TRAF1 and TRAF2 and while they may bind to caspases 7 and 9, they cannot directly inhibit their proteolytic activity. It has therefore been suggested that they might regulate apoptosis indirectly, by influencing signaling pathways elicited by the TNF receptor superfamily.

Natural antagonists of IAPs include Grim in Drosophila and Smac/DIABLO in mammals. These proteins have been shown to bind to the same groove in the BIRs of XIAP as caspases, and can thereby antagonize XIAP anti-caspase activity in vitro. To mimic the cIAP antagonists, compounds have been designed to prevent XIAP from inhibiting caspases, thereby causing cancer cells to undergo apoptosis. Because XIAP inhibits caspases that are activated by Apaf-1 and cytochrome c, but not caspase 8, cell death caused by cIAP antagonists alone would be expected to be relatively unaffected by the caspase 8 inhibitor crmA.

Binding constants (K_{d}) can be measured using fluorescence polarization. Briefly, varying concentrations of an IAP antagonist are mixed with 5 nM fluorescently labeled peptide (e.g., AbuRPF-K(5-Fam)-NH₂) and 40 nM of an IAP-BIR3 for 15 minutes at RT in 100 μL0.1M potassium phosphate buffer, pH 7.5 containing 100 μg/ml bovine γ-globulin. Following incubation, the polarization values (mP) are measured on a Victor²V using a 485 nm excitation filter and a 520 nm emission filter. IC₅₀ values are determined from the plot using nonlinear least-squares analysis using, for example, GraphPad Prism (San Diego, Calif.). The K_{d} values of competitive inhibitors are calculated using the newly derived equation described based upon the measured IC₅₀ values, the K_{d} value of the probe and IAP BIR3 complex, and the concentrations of the protein and probe in the competition assay.

### Example 2

The IAP family of proteins suppresses apoptosis by preventing the activation of procaspases and inhibiting the enzymatic activity of mature caspases. Several distinct mammalian IAPs including XIAP, cIAP-1, cIAP-2, ML-IAP, NAIP (neuronal apoptosis inhibiting protein), Bruce, and survivin, have been identified, and they all exhibit anti-apoptotic activity in cell culture.

Smac is synthesized in the cytoplasm with an N-terminal mitochondrial targeting sequence that is proteolytically removed during maturation to the mature polypeptide and is then targeted to the inter-membrane space of mitochondria.

It has been shown that Smac promotes not only the proteolytic activation of procaspases, but also the enzymatic activity of mature caspase, both of which depend upon its ability to interact physically with IAPs. This N-terminal sequence is essential for binding IAPs and blocking their anti-apoptotic effects.

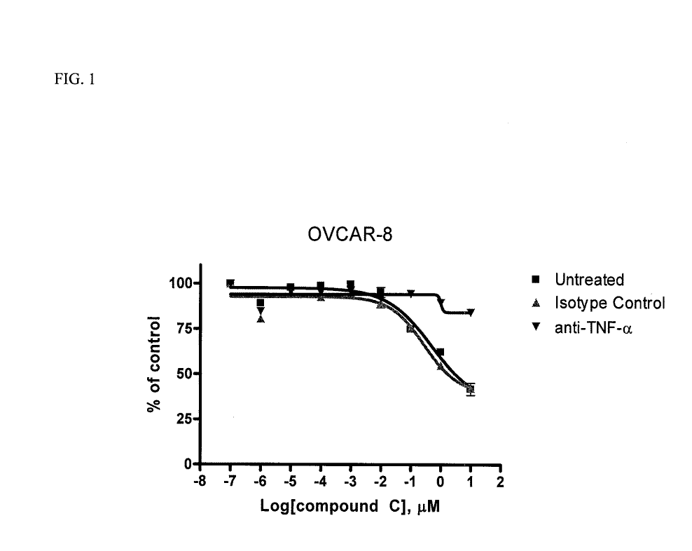

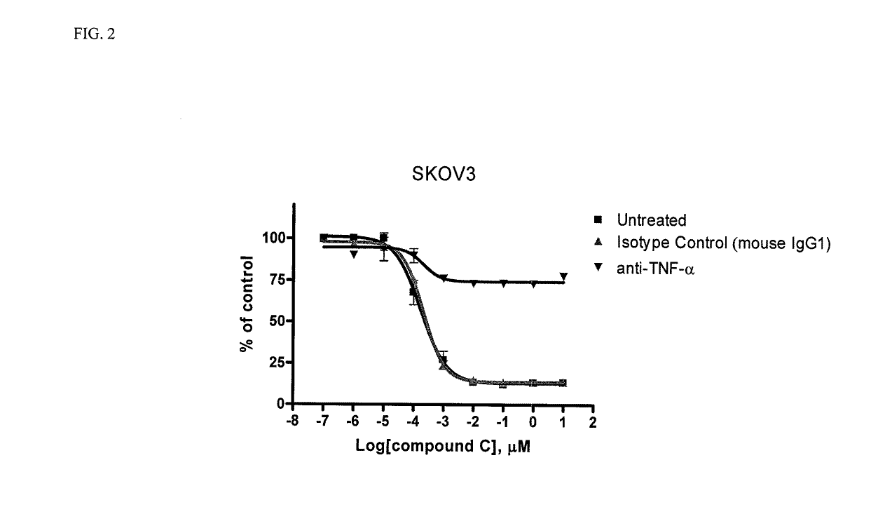

The basic biology of cIAP antagonists suggests that they may complement or synergize with cytokines produced by the cell lines in response to cIAP-1 degradation. We have previously observed that our compounds promote rapid cIAP-1 degradation. Therefore, we hypothesized that degradation of cIAP-1 may result in autocrine activation of the death-receptor pathway by TNFα. To test this, we used TNFα blocking antibody in an MTT assay. Briefly, indicated ovarian cancer cell lines were seeded into 96-well cell culture plates at a density of 5,000 cells per well. The next day, anti-TNFα antibody (MAB610, R&D Systems, Minneapolis, Minn.) was added to cells at a final concentration of 10 g/mL and allowed to incubate for 1 hr at 37° C., 5% CO₂. Following incubation, a range of concentrations of the cIAP antagonist compound C was added to cells. Cells were incubated in the presence of compound C+anti-TNFα antibody or in the presence of compound C alone as a control for an additional 72 hrs. Following 72 hr incubation, MTT reagent (3-(4,5-dimethylthiazolyl-2)-2,5-diphenyltetrazolium bromide; SIGMA, St. Louis, Mo.) was added to all wells at a final concentration of 1 mg/mL and allowed to incubate for 2 hrs at 37° C., 5% CO₂. Following a 2 hr incubation, all media was removed from wells and 50 L DMSO was added to solubilize formazan crystals. After a brief incubation at room temp on a microplate shaker, absorbance was measured at 570 nM using a Victor²V fluorescent microplate reader (Perkin-Elmer, Turku, Finland). Data was converted to percent of untreated controls and CC₅₀ curves were generated using GraphPad PRISM software. FIGS. 1 and 2 show that using an anti-TNFα antibody abrogates cIAP antagonist induced cytotoxicity suggesting TNFα produced by the cells could be synergizing with cIAP antagonists.

TNFα production is a biomarker indicating that a cell or tissue will be sensitive to apoptosis inducing agents such as cIAP antagonists. Furthermore, compound-induced apoptosis was shown to be dependent on autocrine activation of the death-receptor pathway by TNFα. In this case, transcriptional repression of the TNFα gene due to accumulation of mutations in the gene (single nucleotide polymorphisms, SNPs) or by epigenetic means such as promoter methylation represent potential mechanisms that account for resistance of certain tumor cells to cIAP antagonists. Therefore, we propose that analysis of the TNFα promoter region to detect increased methylation and screening the TNFα gene for inactivating mutations provides a rapid means by which patients can be stratified for sensitivity to cIAP antagonists. Analysis of mRNA for presence of TNFα mRNA can also be used. Pre-screening can be performed on tumor biopsies as well as paraffin-embedded tissues obtained from patients and results can be obtained in as little as 3 days as described below:

Methylation Scan of the TNFα promoter

Methylation of Cytosine-Guanine (CpG) dinucleotides in genomic DNA, specifically in the promoter region of the gene, is one of the most frequent epigenetic events leading to transcriptional repression and gene silencing. To detect methylation of the promoter region of the TNF-α gene, genomic DNA is prepared from tumor biopsies as well as normal tissue samples followed by bisulphite conversion and sequencing. This easy method of detection for the presence of methylated cytosines is widely accepted.

Detection of SNPs in TNFα

The small size of the TNF-α gene (2763 bp) permits rapid sequencing of the genomic DNA encoding this gene (GenBank Accession #NC_{—}000006 Region: 31651329 . . . 31654091). Detection of mutation(s) in the TNFα gene can be performed through preparation of genomic DNA from tumor biopsies followed by PCR amplification of the TNFα gene. PCR products are then submitted for nucleotide sequence analysis and compared with sequences obtained from normal tissues.

The invention disclosed herein provides methods and assays examining expression of biomarker in a mammalian tissue or cell sample, wherein the expression of one or more such biomarkers is predictive of whether the tissue or cell sample will be sensitive to cIAP antagonists. The methods and assays examine expression of TNFα.

### Example 3

### Correlation of TNFα protein expression with sensitivity to an IAP antagonist

The following human cancer cell lines were tested: A375 (melanoma), A549 (lung adenocarcinoma), BXPC3 (pancreatic cancer), EVSA-T (breast cancer), HCT-116 (colon cancer), HCT-15 (colon cancer), HT29 (colon cancer), HUVEC (human umbilical vein cells), IGROV1 (ovarian cancer), NCIH 2052 (mesothelioma), NCIH-1975 (lung cancer), Ovcar4 (ovarian carcinoma), Ovcar 8 (ovarian carcinoma), Ovcar3 (ovarian carcinoma), Piedmont 231 (breast), MDA-MB231 (PU-MB231; breast), RPMI7951 (melanoma), RT4 (bladder), SKOV3 (ovarian cancer), T24 (bladder cancer), T98G (glioblastoma), KYM-1 (rhabdosarcoma), and Mia-PaCa (pancreatic cancer).

Cells (1×10⁶) were plated in each well of a 6-well tissue culture dish and grown to confluence. The culture medium was changed, and 100 μl of medium were collected every 24 hours for 3 days. The amount of TNFα protein secreted into the medium was determined using a Human TNF ELISA Kit II (Becton Dickinson Biosciences, catalog No. 550610). Serial dilutions of 500 pg/ml recombinant human TNFα were made to obtain samples (250, 125, 62.5, 31.3, 15.6, and 7.8 pg/ml) to generate a standard curve. The results are shown in FIG. 3, which also indicates the 50% cytotoxic concentration (μM CC₅₀) of IAP antagonist compound C for each cell line. The CC₅₀ was determined by MTT assay, as described in Example 2.

The data shown in FIG. 3 demonstrate that there is a strong correlation between the capacity of cells to secrete TNF-α and sensitivity to IAP antagonists in vitro. See also Table 1, below.

Single Agent Sensitivity (<0.5 μM CC₅₀ by MTT)

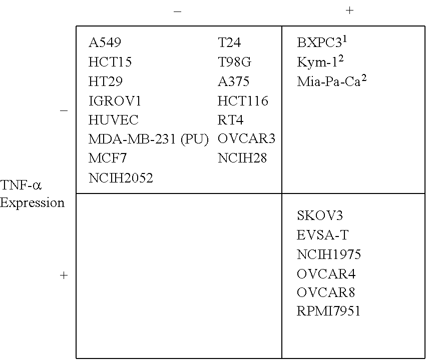

¹TNF-α- and caspase-independent cell death.
²Undergoes apoptosis by TNF-α alone.

Cell lines that responded to IAP antagonist secreted TNFα either in the presence or absence of the IAP antagonist. Resistant cell lines, including HUVEC cells, secreted very little TNFα. Among sensitive cell lines, the amount of secreted TNFα does not directly correlate with the potency of single-agent activity (e.g., EVSA-T vs. SKOV3). Cell lines that are sensitive to TNFα-induced apoptosis (e.g., Kym-1, Mia-Pa-Ca), as expected, do not secrete significant amounts of TNFα. These results support the use of secreted TNFα as a predictive biomarker of sensitivity to IAP antagonists.

1. A method of inducing apoptosis in cells in a cell population, comprising:
assaying a first sample of a cell population in vitro to determine a potential for tumor necrosis factor α gene expression; and
contacting the cell population in vitro with an inhibitor of apoptosis protein antagonist if tumor necrosis factor α gene expression is detected in the first sample.

2. The method of claim 1 wherein the cell population is a cell line.

3. A method of determining sensitivity of cells to an inhibitor of apoptosis protein antagonist, comprising:
assaying cells for a potential for tumor necrosis factor α gene expression; and
identifying the cells as sensitive to an inhibitor of apoptosis protein antagonist if tumor necrosis factor α gene expression is detected.

4. A method of predicting sensitivity of abnormally proliferating cells to treatment with an inhibitor of apoptosis protein antagonist, comprising:
assaying a sample of abnormally proliferating cells for a potential for tumor necrosis factor α gene expression; and
identifying the abnormally proliferating cells as sensitive to treatment with an inhibitor of apoptosis protein antagonist if the cells express the tumor necrosis factor α gene.

5. A method of inducing apoptosis, comprising:
assaying cells of a cell population to determine a potential for tumor necrosis factor α gene expression; and
contacting the cell population with an inhibitor of apoptosis protein antagonist if tumor necrosis factor α gene expression is detected.

6. A method of treating a proliferative disorder, comprising:
(a) sampling pathologically proliferating cells obtained from a patient to determine if the cells have a potential for expressing a tumor necrosis factor α gene; and
(b) administering to the patient
(1) an inhibitor of apoptosis protein antagonist if the potential for expressing the tumor necrosis factor α gene is detected or
(2) an alternative therapy if the potential for expressing the tumor necrosis factor α gene is not detected.

7. A method of screening patients for those who could benefit from treatment with an inhibitor of apoptosis protein antagonist, comprising:
assaying abnormally proliferating cells obtained from a patient for a potential for tumor necrosis factor α gene expression; and
determining that the patient would benefit from treatment with an inhibitor of apoptosis protein antagonist if expression is detected.

8. The method of claim 7 further comprising treating the patient with the inhibitor of apoptosis protein antagonist.

9. A method of determining sensitivity of cells to an inhibitor of apoptosis protein antagonist, comprising:
assaying cells to determine their potential for expressing a tumor necrosis factor α gene in response to NF-κB; and
identifying the cells as sensitive to an inhibitor of apoptosis protein antagonist if a potential for tumor necrosis factor α gene expression is detected.

10. The method of claim 9 wherein the potential for expression of the tumor necrosis factor α gene is assayed by determining the presence of tumor necrosis factor α mRNA in the cell.

11. A method of determining sensitivity of cells to an inhibitor of apoptosis protein antagonist, comprising:
determining if the tumor necrosis factor α gene promoter is methylated; and
identifying the cells as sensitive to an inhibitor of apoptosis protein antagonist if methylation is not detected.

12. A method of determining sensitivity of cells to an inhibitor of apoptosis protein antagonist, comprising:
assaying nuclear factor kappa B response elements within the promoter of the tumor necrosis factor α gene in a sample of cells for mutations; and
identifying the cells as sensitive to an inhibitor of apoptosis protein antagonist if mutations are not detected.

13. The method of claim 1 wherein the cells are selected from the group consisting of tumor cells and cells which abnormally proliferate in an autoimmune disorder.

14. The method of claim 3 wherein the test cells are biopsy cells obtained from a patient.

15. The method of claim 1 wherein cells are contacted with a cytokine or a growth factor before assaying for tumor necrosis factor α gene expression.

16. The method of claim 1 wherein tumor necrosis factor α gene expression is determined by detecting tumor necrosis factor α protein.

17. The method of claim 1 wherein gene expression is determined by detecting tumor necrosis factor α mRNA.

18. The method of claim 1 wherein the inhibitor of apoptosis protein antagonist has a binding affinity for at least one of cellular inhibitor of apoptosis protein 1 and cellular inhibitor of apoptosis protein 2 which is greater than the binding affinity of the cellular inhibitor of apoptosis protein antagonist for X-linked inhibitor of apoptosis protein.

19. The method of claim 1 wherein the binding affinity of the inhibitor of apoptosis antagonist is at least 3-fold greater for cellular inhibitor of apoptosis protein 1 than for X-linked inhibitor of apoptosis protein.

20. The method of claim 1 wherein the binding affinity of the inhibitor of apoptosis protein antagonist for cellular inhibitor of apoptosis protein 1 is at least 100 times greater than for X-linked inhibitor of apoptosis protein.

21. The method of claim 1 wherein the inhibitor of apoptosis protein antagonist is a cellular inhibitor of apoptosis protein antagonist and an X-linked inhibitor of apoptosis protein antagonist.

22. The method of claim 1 wherein the inhibitor of apoptosis protein antagonist has a binding affinity for X-linked inhibitor of apoptosis protein that is greater than the binding affinity of the inhibitor of apoptosis protein antagonist for at least one of cellular inhibitor of apoptosis protein 1 and cellular inhibitor of apoptosis protein 2.

23. (canceled)
